(19) United States
(12) Patent Application Publication
(10) Pub. No.: US 2018/0147111 A1
(43) Pub. Date: May 31, 2018

(54) MEDICAL PROTOCOL FOR IMPROVING HUMAN SEXUAL FUNCTION

(71) Applicant: The Anti-Aging Group, LLC, Aventura, FL (US)

(72) Inventor: Richard Gaines, Aventura, FL (US)

(21) Appl. No.: 15/823,244

(22) Filed: Nov. 27, 2017

Related U.S. Application Data
(60) Provisional application No. 62/426,377, filed on Nov. 25, 2016.

(51) Int. Cl. A61H 19/00; A61H 23/00; A61K 35/19

(52) U.S. Cl. CPC A61H 19/32; A61H 23/008; A61K 9/0026; A61H 19/34; A61K 35/19

(57) ABSTRACT

A medical procedure for improving human sexual function in a patient is disclosed. The medical procedure includes drawing blood from the patient, processing the blood that was drawn to produce platelet rich plasma (PRP) from said blood, and anesthetizing a portion of a penile shaft of a male patient or anesthetizing a portion of a vulva and an anterior vaginal wall of a female patient. The medical procedure also includes applying extra corporeal shockwave therapy to portions of the penis (in a male patient) or portions of the vulva (in a female patient) using an extra corporeal shockwave generator system, and injecting the platelet rich plasma produced from said blood into at least one location on the perineal area of the patient.

### CROSS-REFERENCE TO RELATED APPLICATIONS

This patent application claims priority to provisional patent application No. 62/426,377 filed Nov. 25, 2016 and entitled “Medical Protocol for Improving Human Sexual Function. Provisional patent application No. 62/426,377 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The technical field relates generally to medicine and, more specifically, to methods and procedures for improving human sexual function.

### BACKGROUND

The importance of maintaining good sexual health has been clearly demonstrated in the medical literature. Worldwide, well over 50% of men over 40 years of age have some form of sexual dysfunction. As sexual function deteriorates, the incidence of other diseases, such as cardiovascular disease, diabetes, and depression increases. As the incidence of said diseases increases, so does the requirement for pharmaceutical medications to treat said diseases. Certain medications, such as anti-depressants, can impede sexual function, which compounds the problem for patients.

Conventional existing therapies for treating sexual function are lacking in a variety of ways. Psychological counseling aids in treating the psychological aspect of sexual dysfunction, but does not address the physical aspect of the affliction, and therefore has met with mixed results. Oral medications, such as Viagra, Cialis, Stendra and Levitra, have had success in treating sexual dysfunction, but said medications must be taken each time sexual activity is expected, which may provide unsatisfactory results in many patients. Various companies have asserted that certain herbal and non-FDA approved substances can aid in sexual dysfunction, but there currently is not enough medical evidence to support these claims. Injectable penile medications, such as Trimix and Caverjet, have also had success in treating persons with erectile dysfunction, but these solutions require that medicine is injected directly into the penis, which can be painful and has its own side effects. Yet another approach to sexual dysfunction is the use of penile pump vacuum devices, which are administered to the penis just before sexual activity. Penile pump vacuum devices, however, can be unwieldy and uncouth to utilize before sexual activity in the long term, which is not desirable for many patients. Surgical penile implants have also been used to treat sexual dysfunction, but the cost, invasiveness, and possible serious complications of the surgery are prohibitive for many consumers. Lastly, platelet rich plasma procedures have also been used to treat sexual dysfunction. Platelet-rich plasma (PRP) is blood plasma that has been enriched with platelets, and as a concentrated source of autologous platelets, PRP contains several different growth factors and other cytokines that can stimulate healing of tissue. The use of PRP procedures by themselves, however, have had mixed results in the treatment for sexual dysfunction.

Therefore, a need exists for improvements over the prior art, and, more particularly, for safer and more effective non-invasive methods and therapies for treating sexual dysfunction, or simply improving sexual function, in patients.

### SUMMARY

An improved method and procedure for improving sexual function in patients is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a medical procedure for improving human sexual function in a male patient includes drawing blood from the patient; processing the blood that was drawn to produce platelet rich plasma (PRP) from said blood; anesthetizing a portion of a penile shaft of the patient; applying extra corporeal shockwave therapy to a penis of the patient using an extra corporeal shockwave generator system; and injecting the PRP produced from said blood into at least one location on the perineal area of the patient.

In another embodiment, a medical procedure for improving human sexual function in a female patient includes drawing blood from the patient; processing the blood that was drawn to produce PRP from said blood; anesthetizing a portion of a vulva and an anterior vaginal wall of the patient; applying extra corporeal shockwave therapy to a vulva of the patient using an extra corporeal shockwave generator system; and injecting the PRP produced from said blood into at least one location on the perineal area of the patient.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a flowchart showing the steps of the medical procedure for improving human sexual function in a patient, according to an example embodiment.

FIG. 2 is an illustration of a system used for a medical procedure for improving human sexual function in a patient, according to one embodiment.

FIG. 3 is an illustration of a frontal cross sectional view of a penis, showing its interior organs, according to one embodiment.

FIG. 4 is an illustration of a perspective side view of a penis, showing its interior organs, according to one embodiment.

FIG. 5 is an illustration of a front view of the perineal area of a female patient, showing its interior organs, according to one embodiment.

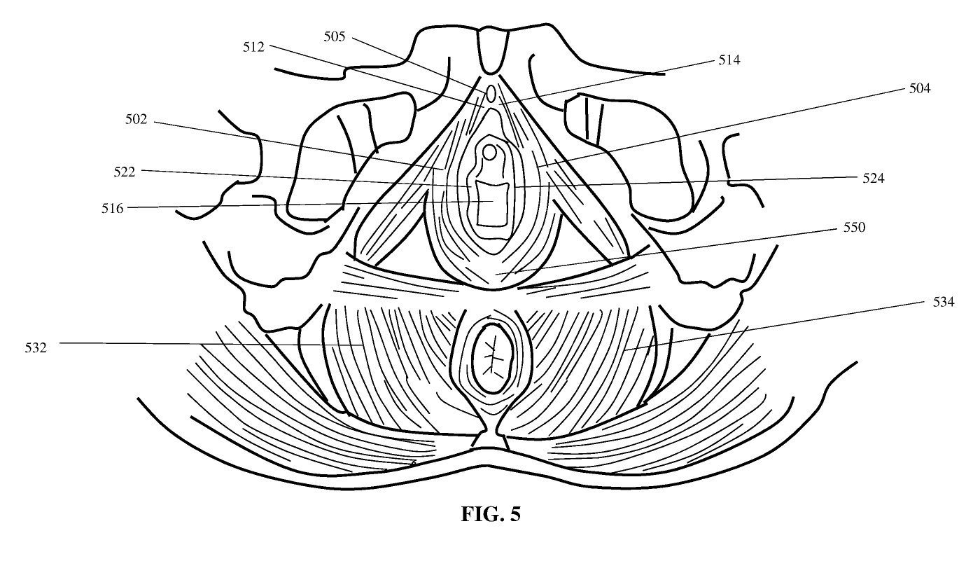

### DETAILED DESCRIPTION

The following detailed description refers to the accompanying drawings. Wherever possible, the same reference numbers are used in the drawings and the following description to refer to the same or similar elements. While embodiments herein may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves over the prior art by providing a more efficient, safe and precise way of treating sexual dysfunction in individuals and/or improving human sexual function. The claimed subject matter utilizes a new method of treating sexual dysfunction and improving human sexual function that includes the use of platelet rich plasma (PRP) procedures, extracorporeal shock wave therapy, sexual aid devices (such as penile vacuum pumps and vibrators) and various medications. The claimed subject matter provides long lasting results in treating sexual dysfunction and improving human sexual function without the need for invasive surgery, the use of expensive machinery or the need for medications that can have serious side effects. The claimed subject matter is further easy to administer to patients in an outpatient manner and simple to understand for patients. The result of the claimed medical procedures is improved sexual performance and sexual function.

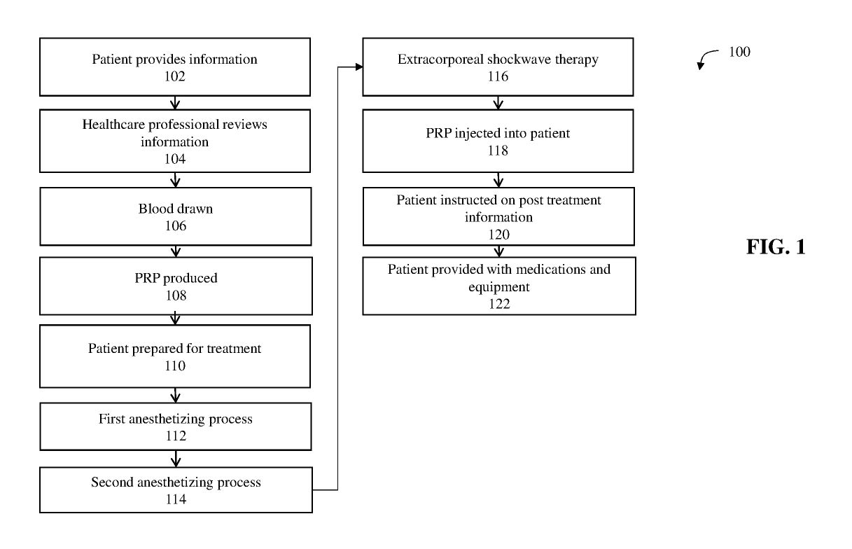

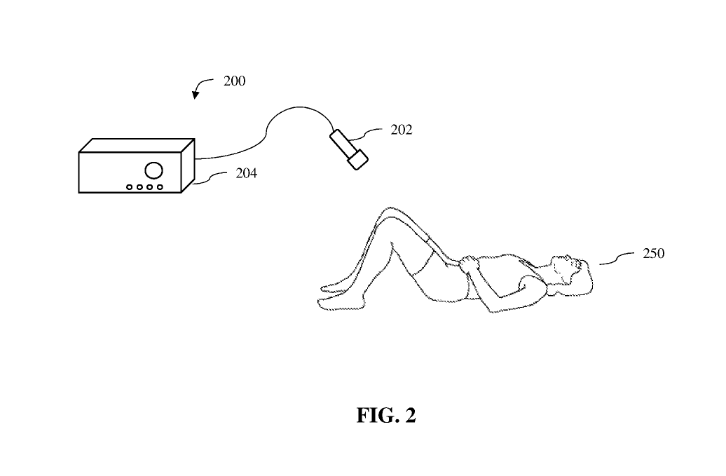

FIG. 1 is a flow chart showing the steps of the medical procedure 100 for improving human sexual function in a patient, according to an example embodiment. Process 100 describes the steps that occur when a healthcare professional engages in the medical procedure 100 for improving human sexual function in a patient. The process 100 is described with reference to FIG. 2, which shows an illustration of a system 200 used for the medical procedure. Initially, in an outpatient setting, such as a clinic or doctor's office,

In a first step 102, a patient is asked to fill out a medical history document, and answer a medical questionnaire. For a male patient, a Sexual Health Inventory for Men (SHIM) questionnaire, an Erectile Hardness Score questionnaire and a global questionnaire may be used, all of which are designed to help the attending healthcare professional identify if the patient may be experiencing erectile dysfunction. A SHIM score may be obtained from the SHIM Questionnaire and an Erectile Hardness Score may be obtained from the Erectile Hardness Score questionnaire. In a female patient, a Female Sexual Function Index (FSFI) questionnaire may be used, which evaluates the sexual function of the patient, as well as the level of dysfunction, if any. A FSFI score may be obtained from the FSFI questionnaire. Next, in step 104 the attending healthcare professional will review the results of the medical history document, the questionnaires, and the resulting scores and perform a directed physical exam of the patient. The claimed medical procedure is then discussed in detail with the patient and the risks and benefits of the procedure are presented.

In step 106, a predefined amount of blood, such as 60 cc, is drawn off from the patient using an aseptic technique. A local anesthetic may be injected into the patient, or a topical anesthetic may be applied, for the blood draw. The drawn 60 cc of blood is placed into a kit and placed into a specialized centrifuge, so as to undergo the process, in step 108, for producing platelet rich plasma (PRP) from the drawn blood, which is well known in the art. Techniques for producing platelet-rich plasma include a two-step centrifugation method and a separation system, such as a gravitational platelet separation system.

In step 110, the patient is instructed to disrobe from waist down, the patient is covered with a disposable sheet, and the patient is placed supine on the procedure table, as shown in FIG. 2. Next, in step 112, a first anesthetizing process may be used. In a male, a topical anesthetic, such as a creme (well known in the art), may be applied to the shaft of the penis and the perineal region (bulbospongiosal region). The anesthetic may be applied to portions of the penile shaft and portions of the perineal region of the patient, or to the entire penile shaft and the entire perineal region of the patient. The anesthetic may further be applied directly at or near the left or right pudendal canals of the male patient. In a female, the topical anesthetic may be separately applied to the vulva, the anterior vaginal wall and the clitoral regions, as well as the perineal region. The anesthetic may be applied to portions of the vulva, portions of the clitoral region, and portions of the perineal region of the patient, or to the entire vulva, the entire clitoral region and the entire perineal region of the patient. The anesthetic may further be applied directly at or near the left or right pudendal canals of the female patient. For subsequent treatments, the patient may himself or herself apply the topical anesthetic 30 minutes prior to arrival to the outpatient setting. Alternative to the topical anesthetic, a local anesthetic well known in the art may be used. The perineum is defined as the surface region in both males and females that is between the thighs and includes the urogenital triangle and the anal triangle, i.e., it includes the entirety of the genitals in both males and females, as well as the anus.

Then, in step 114, a second, optional anesthetizing process may be used. In a male, the penile shaft may anesthetized with a 2% lidocaine solution without epinephrine, wherein 0.5 to 2.0 ml lidocaine is injected over the dorsal penile nerves. The lidocaine solution may further be applied to strategic locations in the perineal region (bulbospongiosal region), and the left or right pudendal canals of the male patient. In a female, the vulva may be anesthetized with a 2% lidocaine solution without epinephrine, wherein 0.5 to 2.0 ml lidocaine is injected at or near the dorsal clitoral nerve, such as at the junction of the labia minora and the clitoral hood (512, 514). The lidocaine solution may further be applied to strategic locations in the perineal region, the clitoral region, the vulva, and the left or right pudendal canals of the female patient.

After adequate anesthesia of the genital area is assured, in step 116, extracorporeal shock wave therapy is applied uniformly over strategic regions or portions of the patient. Extracorporeal shock wave therapy is a treatment used to treat various maladies. Extracorporeal shock wave therapy is known to produce revascularization via angiogenesis and has been used in other countries for many years in the treatment of various conditions from poor wound healing in diabetics to plantar fasciitis.

Shockwaves, which are abrupt, high amplitude pulses of mechanical energy, similar to soundwaves, are generated by an electromagnetic or hydroelectric force, and applied to the patient. The term “extracorporeal” means that the shockwaves are generated externally to the body and transmitted from a coupling medium through the skin. Extracorporeal shock wave therapy, which is FDA approved, is applied using an extracorporeal shock wave generator system 200 shown in FIG. 2. Shockwaves stimulate angiogenesis (new blood vessels) and neurogenesis (new nerve cells). The cells are stimulated, which promotes the release of growth factor and cytokines that are associated with removing damaged matrix constituents, and stimulates wound healing mechanisms.

The extracorporeal shock wave generator system 200 shown in FIG. 2 comprises a shock wave generator 204 and a shockwave applicator 202, which is directly applied to the patient 250 by a healthcare professional.

Various variables can be selected during the application of extracorporeal shock wave therapy to the patient. This includes the selection of the bar value (which refers to the pressure magnitude of the shockwaves), the frequency of the shockwaves, the number of pulses and the type of tip used on the applicator when applying the extracorporeal shock wave therapy to the patient. For a particular patient, the bar value, frequency, number of pulses and tip type is selected in a fashion such that the most ideal force that can be tolerated by the patient (approximately 2.6-3.4 bar, resulting in 0.09-0.30 mJ/mm² or 0.19-0.30 mJ/mm² of pressure). These details are recorded so that appropriate settings can be applied in subsequent sessions to said patient. The more ideal the bar value tolerated by the patient may be, the more effective the tissue stimulation will be achieved during the procedure. After extracorporeal shock wave therapy is complete, the surface skin of the patient may be pink or reddened after the procedure, due to increased blood flow to the area. This is a transient effect and should disappear shortly after extracorporeal shock wave therapy is complete.

With regard to the extracorporeal shock wave therapy, the frequency of the shockwave may range from a low of 12 Hz to a high of 19 Hz, the bar value of the shockwave may range from a low of 2.6 bar to a high of 3.4 bar, and the number of pulses may range from 5,000 to 16,000, with a preferred range of 8,000-14,000. The healthcare professional may begin the extracorporeal shock wave therapy with a high frequency and a low bar pressure (such as 2.6 bar, 19 Hz, for 2,000 pulses, for example), and proceed to a low frequency and a high pressure (such as 3.4 bar, at 12-15 Hz, for 2,000 pulses, for example). This is because a low bar value and a high frequency are often associated with less pain in the patient, while a high bar value and a low frequency often associated with greater pain in the patient. Thus, the healthcare professional may start with a low bar value and a high frequency and slowly increase the bar value while lowering the frequency, for best results. Bar values may be measured in bars and in mJ/mm², as applied to the tissue level, which means that the pressure is applied to the surface of the patient's skin via a coupling agent, such as an ultrasound gel, which insures adequate energy delivery to the strategic underlying tissues.

Recall that the extracorporeal shock wave generator system 200 shown in FIG. 2 comprises a shock wave generator 204 and a shockwave applicator 202, which is directly applied to the patient 250 by a healthcare professional. In one embodiment, a topical anesthetic is applied to the applicator 202 as a coupling agent, so as to lessen the pain experienced by the patient, and later a conventional coupling agent, such as ultrasound gel, may be applied. The topical anesthetic may delivered to, and act upon, the underlying tissue via sonoporation.

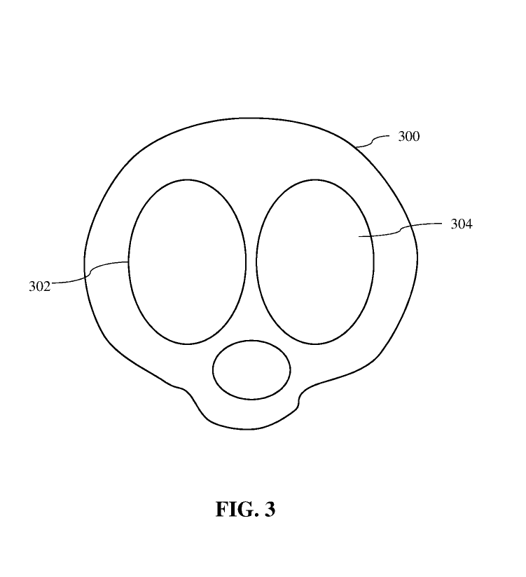

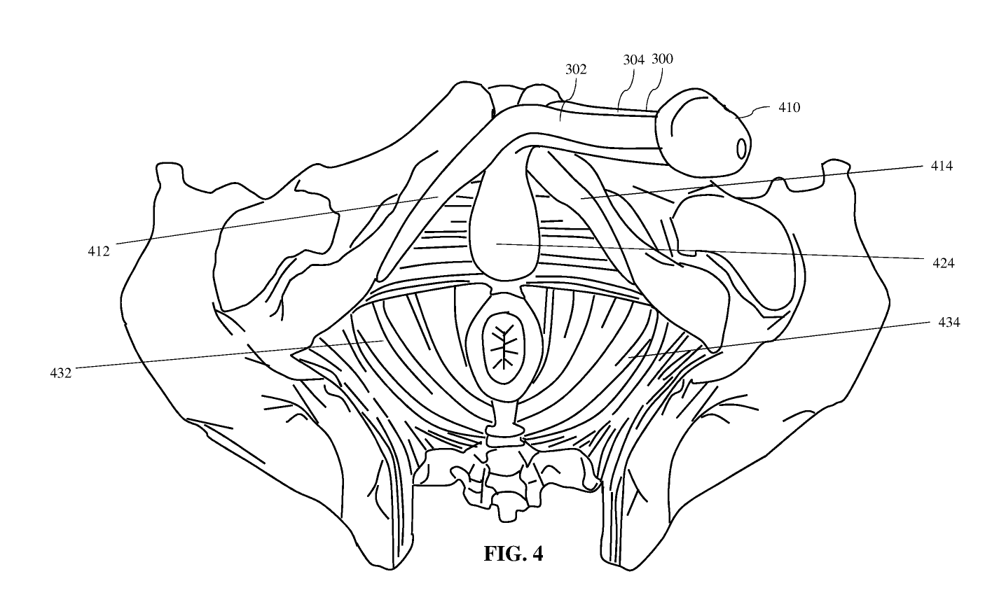

In a male patient, extracorporeal shock wave therapy may be applied to the following strategic locations on the patient's body using the shockwave applicator 202:
a. the right corpus cavernosum 302 (see FIG. 3) of the penis 300, along the penile shaft;
b. the left corpus cavernosum 304 of the penis 300, along the penile shaft;
c. the right crura 412 (see FIG. 4);
d. the left crura 414;
e. the bulbospongiosum 424;
f. the right side perineal or pudendal neurovascular bundle 432;
g. the left side perineal or pudendal neurovascular bundle 434.

A corpus cavernosum is one of a pair of sponge-like regions of erectile tissue, which contains most of the blood in the penis during an erection. Each corpus cavernosum extends downwards towards the ischial arch and ends at an extension referred to as a crura. The bulbospongiosum 424 is erectile tissue located at the base of the penile shaft above the anus, wherein the bulbospongiosum extends upwards and ends in the glans. The perineal or pudendal neurovascular bundle is a neurovascular bundle comprised of a combination of nerves, arteries, veins, and lymphatics that extend together through the pudendal canal and innervate the pelvic floor and the perineum. The pudendal canal carries the pudendal nerve. In the event that certain anatomical structures are located internally, i.e., under the patient's skin, such as the right crura, the application of the extracorporeal shock wave therapy is effectuated by applying the shockwave applicator 202 to the patient's skin directly above said anatomical structure.

In a female patient, extracorporeal shock wave therapy may be applied to the following strategic locations on the patient's body using the shockwave applicator 202:
a. the clitoris 505 or the lateral portions (512, 514) of the clitoris (such as the junction of the labia minora and the clitoral hood);
b. the left labia minora 524 and left labia majora 504;
c. the right labia minora 522 and right labia majora 502;
d. the anterior vaginal wall 516 or the entire interior circumference of the distal vaginal canal;
e. the right side perineal or pudendal neurovascular bundle 532;
f. the left side perineal or pudendal neurovascular bundle 534;
g. perineal raphe 550.

The anterior vaginal wall 516 is the wall of the interior vaginal canal that is closest to the front side or anterior side of the patient's body. The extracorporeal shock wave therapy is applied to the anterior vaginal wall by covering the shockwave applicator 202 with a rubber cover, such as a condom, and inserting the applicator into the vaginal canal. Alternatively, the extracorporeal shock wave therapy may be applied to the entire interior circumference of the distal portion (i.e., lower portion) of the vaginal canal using the shockwave applicator 202. The perineal raphe is a ridge of tissue on the human body that extends from the anus to the opening of the vagina, in a female. In the event that certain anatomical structures are located internally, i.e., under the patient's skin, such as the pudendal neurovascular bundle, the application of the extracorporeal shock wave therapy is effectuated by applying the shockwave applicator 202 to the patient's skin directly above said anatomical structure.

Next, in step 118, the patient is prepared for injection of the PRP that was generated, and the patient is subsequently injected as described herein. In addition to the PRP, the injections used on the patient may also contain additional medications or drugs, including cross-linked hyaluronic acid, human growth hormone, peptides, such as thymosin beta 4, bone marrow aspirate and stromal vascular fraction (SVF)/tissue vascular fraction (TVF) derived from adipose tissue of the patient. All injections are performed with an aseptic technique after adequate anesthesia is established.

In a male patient, multiple 5 ml syringes are prepared with 4 ml of PRP each and a single 1 ml syringe is prepared with 1 ml of PRP. Subsequently, injections are made into the patient with a 25-27 gauge short needle (such as a ½ inch needle) as follows:
a. 2 ml of PRP are injected into a first site of the right corpus cavernosum 302 (see FIG. 3 and FIG. 4) of the penis 300, preferably along the distal third of the length of the penile shaft;
b. 2 ml of PRP are injected into a second site of the right corpus cavernosum 302 of the penis 300, preferably along a proximal third of the length of the penile shaft;
c. 2 ml of PRP are injected into a first site of the left corpus cavernosum 304 of the penis 300, preferably along the distal third of the length of the penile shaft;
d. 2 ml of PRP are injected into a second site of the left corpus cavernosum 304 of the penis 300, preferably along the proximal third of the length of the penile shaft;
e. 1 ml of PRP is injected into the glans 410 (see FIG. 4) of the penis.

In a female patient, multiple 3 ml or 5 ml syringes are prepared with 3-5 ml of PRP. Subsequently, injections are made into the patient with a 25-27 gauge short needle (such as a ½ inch needle) as follows:
a. 3-5 ml of PRP are injected into the anterior vaginal wall 516, using a 25-27 gauge, 1 and ¼ inch needle;
b. 1-2 ml of PRP are injected into a lateral portion (512, 514) of the clitoris (such as the junction of the labia minora and the clitoral hood);
c. optionally, 2-4 ml of PRP are injected into the right labia majora 502;
d. optionally, 2-4 ml of PRP are injected into the left labia majora 504.

Additional substances may be injected into the patient separately from the PRP, including cross-linked hyaluronic acid, human growth hormone, peptides, such as thymosin beta 4, and SVF/TVF derived from adipose tissue of the patient.

Then, in step 120, the patient is instructed on a variety of post treatment information. For a male patient, the application of a penile vacuum pump, vibrator or other sexual aid device is instructed. Use of a penile vacuum pump is demonstrated to the patient by applying the penile vacuum pump to the penile shaft of the patient and pumping the penile vacuum pump to induce an erection in the patient. Post procedure instructions are given verbally and in written form. The male patient is instructed to pump his penis twice daily for 10 minutes. The male patient is instructed to pump the penile vacuum pump until it reads −10 mm Hg or 35-40 kPa in pressure, as tolerated. The male patient is instructed to repeat this procedure each day for 30 days and then once weekly after that Penile traction therapy is also discussed with the male patient. Penile traction therapy uses mechanical forces to lengthen or straighten the penis. In a female patient, the use of a vibrator, prosthetic penis, clitoral stimulator device, clitoral suction device or other sexual aid device may be instructed. The patient is instructed to use said sexual aid devices as needed.

In step 122, the patient is instructed on the use of, and provided with, medications, such as an apomorphine medication or a PDE5 inhibitor, such as tadalafil, avanafil, and sildenafil. The patient may be provided with one or more troches that include 8 doses of medication that are to be taken every other night for the following 16 days.

The patient is told he or she may experience possible bruises, which are slight and transient. The patient is also instructed not to take anti-inflammatory medications such as aspirin, acetylsalicylic acid, or ibuprofen, for 3 days before the procedure and 7 days after the procedure. Acetaminophen or opiates can be used if necessary for other pain conditions. The patient is instructed that normal sexual activities can be resumed within 24 hours after the procedure.

In one embodiment, the treatment described in process 100 is provided to the patient twice a week for 3 weeks, for a total of 6 treatments. In another embodiment, the number of treatments is based on the level of dysfunction, such as 6 treatments for mild erectile dysfunction and 12 treatments for severe dysfunction. In yet another embodiment, the extracorporeal shockwave therapy step 116 is performed two Limes on separate visits before the entire process 100 is executed all at once in one visit.

### Experimental Results

In one study conducted over 2016-2017, 112 human patients were treated using the claimed treatment. Prior to the study, in the population of said 112 patients: 52% wanted to improve sexual performance, 42% wanted to treat erectile dysfunction, 41% use PDE5s (of which 39% responded poorly), 20% had very low confidence in achieving or maintaining an erection and 28% sometimes had erections hard enough for penetration. After three applications of the claimed treatment, the following results were recorded: 55% of the patients stated that the treatment improved their erectile functionality, 30% stated they have been able to stop PDE5s, 46% stated the PDE5s work better for them, only 14% have low confidence in achieving or maintain erection, and 35% stated that their erection was hard enough for penetration. After five applications of the claimed treatment, the following results were recorded: 70% of the patients stated that the treatment improved their erectile functionality, 39% stated they have been able to stop PDE5s, 60% stated the PDE5s work better for them, 39% have a high confidence in achieving or maintain erection, and 27% stated that their erection was hard enough for penetration always. The conclusion of said study is that the results are encouraging and indicative of success. The claimed treatment is shown to boost confidence and increase erection functionality, helps patients either respond better or discontinue use of PDE5s, and there are no significant adverse effects reported to date.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A medical procedure for improving human sexual function in a male patient, said medical procedure comprising:
(a) drawing blood from the patient;
(b) processing the blood that was drawn to produce platelet rich plasma (PRP) from said blood;
(c) anesthetizing a portion of a penile shaft of the patient;
(d) applying extra corporeal shockwave therapy to portions of a penis of the patient using an extra corporeal shockwave generator system; and
(e) injecting the platelet rich plasma produced from said blood into at least one location on a perineal area of the patient.

2. The medical procedure of claim 1, wherein the step of drawing blood from the patient further comprises drawing a predefined amount of blood from the patient.

3. The medical procedure of claim 2, wherein the step of processing the blood further comprises processing the blood that was drawn to produce PRP from said blood, wherein an amount of PRP that is produced is based upon the predefined amount of blood.

4. The medical procedure of claim 3, wherein the step of anesthetizing a portion of the penile shaft of the patient further comprises applying a local anesthetic to a portion of the penile shaft of the patient.

5. The medical procedure of claim 3, wherein the step of anesthetizing a portion of the penile shaft of the patient further comprises applying a topical anesthetic to a portion of the penile shaft of the patient.

6. The medical procedure of claim 3, wherein a portion of the penile shaft of the patient comprises an entirety of the penile shaft of the patient.

7. The medical procedure of claim 3, wherein the step of applying extra corporeal shockwave therapy to portions of the penis of the patient further comprises setting the extra corporeal shockwave generator system to a bar value that delivers between 0.09-0.30 mJ/mm² at a tissue level of a target area.

8. The medical procedure of claim 3, wherein the step of applying extra corporeal shockwave therapy to the portions of the penis of the patient further comprises setting the extra corporeal shockwave generator system to a bar value that delivers between 0.19-0.30 mJ/mm² at a tissue level of a target area.

9. The medical procedure of claim 3, wherein the step of injecting the PRP produced from said blood further comprises injecting the PRP produced from said blood into a plurality of locations on the perineal area of the patient.

10. The medical procedure of claim 1, wherein the medical procedure further includes an additional step of anesthetizing portions of a perineal area of the patient.

11. The medical procedure of claim 10, wherein the step of anesthetizing a portion of the perineal area of the patient further comprises applying a local anesthetic or a topical anesthetic to a portion of the perineal area of the patient.

12. The medical procedure of claim 11, wherein a portion of the perineal area of the patient comprises an entirety of the perineal area of the patient.

13. A medical procedure for improving human sexual function in a female patient, said medical procedure comprising:
(a) drawing blood from the patient;
(b) processing the blood that was drawn to produce platelet rich plasma (PRP) from said blood;
(c) anesthetizing a portion of a vulva and an anterior vaginal wall of the patient;
(d) applying extra corporeal shockwave therapy to portions of the vulva of the patient using an extra corporeal shockwave generator system; and
(e) injecting the platelet rich plasma produced from said blood into at least one location on the perineal area of the patient.

14. The medical procedure of claim 13, wherein the step of drawing blood from the patient further comprises drawing a predefined amount of blood from the patient.

15. The medical procedure of claim 14, wherein the step of processing the blood further comprises processing the blood that was drawn to produce PRP from said blood, wherein an amount of PRP that is produced is based upon the predefined amount of blood.

16. The medical procedure of claim 15, wherein the step of anesthetizing a portion of the vulva and the anterior vaginal wall of the patient further comprises applying a local anesthetic to a portion of the vulva and the anterior vaginal wall of the patient.

17. The medical procedure of claim 15, wherein the step of anesthetizing a portion of the vulva and an anterior vaginal wall of the patient further comprises applying a topical anesthetic to a portion of the vulva and the anterior vaginal wall of the patient.

18. The medical procedure of claim 15, wherein a portion of the vulva of the patient comprises an entirety of the vulva of the patient.

19. The medical procedure of claim 15, wherein the step of applying extra corporeal shockwave therapy to portions of the vulva of the patient further comprises setting the extra corporeal shockwave generator system to a bar value that delivers between 0.09-0.30 mJ/mm² at a tissue level of a target area.

20. The medical procedure of claim 15, wherein the step of applying extra corporeal shockwave therapy to portions of the vulva of the patient further comprises setting the extra corporeal shockwave generator system to a bar value that delivers between 0.19-0.30 mJ/mm² at a tissue level of a target area.

21. The medical procedure of claim 15, wherein the step of injecting the PRP produced from said blood further comprises injecting the PRP produced from said blood into a plurality of locations on the perineal area of the patient.

22. The medical procedure of claim 13, wherein the medical procedure further includes an additional step of anesthetizing portions of a perineal area of the patient.

23. The medical procedure of claim 22, wherein the step of anesthetizing a portion of the perineal area of the patient further comprises applying a local anesthetic or a topical anesthetic to a portion of the perineal area of the patient.

24. The medical procedure of claim 23, wherein a portion of the perineal area of the patient comprises an entirety of the perineal area of the patient.
